(19) United States
(12) Patent Application Publication
(10) Pub. No.: US 2019/0117434 A1
(43) Pub. Date: Apr. 25, 2019

(54) FIXATION DEVICE FOR PELVIS

(71) Applicant: Xieping ZHU, Shanghai (CN)

(72) Inventor: Xieping ZHU, Shanghai (CN)

(21) Appl. No.: 15/790,082

(22) Filed: Oct. 23, 2017

(51) Int. Cl. A61F 5/05; A61F 5/02; A61F 13/04

(52) U.S. Cl. CPC A61F 5/05; A61F 5/028; A61F 2013/00578; A61F 13/048; A61F 5/022

(57) ABSTRACT

A fixation device for pelvis includes a front fixation plate and a back fixation plate. The front fixation plate and the rear fixation plate are detachably connected to form a receiving space for receiving the waist, the abdomen, the hip and the thighs. The front fixation plate includes a first left-side piece and a first right-side piece. The rear fixation plate includes a second left-side piece and a second right-side piece. The first left-side piece, the first right-side piece, the second left-side piece and the second right-side piece are respectively provided with a cavity for accommodating a polyurethane slurry. When in use, the cavities are respectively injected with the polyurethane slurry, and after the polyurethane slurry solidifies, the shape of the fixation device is matched to the waist, the abdomen, the hip and the thighs to support the patient's hip and pelvis.

### BACKGROUND OF THE INVENTION

### Field of the Invention

The invention relates to a fixation device for pelvis.

### Description of the Related Art

The number of pelvic fractures increases from year to year. Conventional devices for treating pelvic fracture include splints, plasters, external fixators, and traction fixators. However, all such devices are not ideal. For example, splints are likely to result in bone displacement; plasters do not allow for convenient monitoring and treatment of injured soft tissue; and external fixators hinder rehabilitation.

### SUMMARY OF THE INVENTION

In view of the above-described problems, one objective of the invention is to provide a fixation device for pelvis which is easy to use, provides satisfactory fixation, can fully meet the treatment needs, can effectively support and fix fractures, and can shorten the period of treatment and rehabilitation.

To achieve the above objective, in accordance with one embodiment of the invention, there is provided a fixation device for pelvis, comprising: (a) a front fixation plate comprising a first left-side piece and a first right-side piece; the first left side piece comprises a first cavity and a first feeding port; the first right-side piece comprises a second cavity and a second feeding port; and (b) a rear fixation plate comprising a second left-side piece and a second right-side piece; the second left-side piece comprises a third cavity and a third feeding port; the second right-side piece comprises a fourth cavity and a fourth feeding port. The front fixation plate is used to cover the abdomen, the front side of the waist and thighs of the patient; and the rear fixation plate is used to cover the hip and the back side of the waist of the patient. The front fixation plate and the rear fixation plate are detachably connected to each other to form a space for receiving the waist, hip and the thighs. The first left-side piece and the first right-side piece are connected to each other at a position corresponding to the waist and the abdomen; the second left-side piece and the second right-side piece are connected at a position corresponding to the waist and the hip. The first, the second, the third and the fourth cavities are respectively disposed on the first left-side piece, the first right-side piece, the second left-side piece and the second right-side piece. The first, second, third and fourth feeding ports are respectively disposed on the first left-side piece, the first right-side piece, the second left-side piece and the second right-side piece, and respectively communicates with the first, second, third and fourth cavities. When in use, the first, the second, the third and the fourth cavities are filled with polyurethane slurry through the first, second, third and fourth feeding ports; and after the polyurethane slurry enters the solid phase stage, the shapes of the first left-side piece, the first right-side piece, the second left-side piece and the second right-side piece are matched to the waist, the abdomen, the thighs, and the hip.

In a class of this embodiment, the fixation device further comprises a detachable connector. The first left-side piece and the first right-side piece are respectively detachably connected to each other at positions corresponding to the inner and outer sides of the thighs through the detachable connector. In addition, the second left-side piece and the second right-side piece are respectively detachably connected to each other at positions corresponding to the inner and outer sides of the thighs through the detachable connector.

In a class of this embodiment, the detachable connector is a zipper, a snap fastener or a hook-and-loop fastener.

In a class of this embodiment, the first, the second, the third and the fourth feeding ports are respectively provided with a hook-and-loop fastener.

In a class of this embodiment, the first left-side piece, the first right-side piece, the second left-side piece, and the second right-side piece respectively comprises two fabric layers; the two fabric layers are overlapped; and the first, second, third and fourth cavities are formed between the two fabric layers.

In a class of this embodiment, the first left-side piece is sewed to the first right-side piece so that the first cavity does not communicate to the second cavity; the second left-side piece is sewed to the second right-side piece that the third cavity does not communicate with the fourth cavity.

In a class of this embodiment, the first, the second, the third and the fourth feeding ports are respectively disposed on the first left-side piece, the first right-side piece, the second left-side piece, and the second right-side piece at positions corresponding to the waist.

The fixation device is easy to use, can quickly form a shape matched with the body, provides satisfactory fixation effect, fully meets the doctor's needs for treating the patients, prevents the rigidity or adhesions of the joint tissues, and shortens period of the treatment and rehabilitation.

### BRIEF DESCRIPTION OF THE DRAWINGS

FIG. 1 is a perspective view of a fixation device for pelvis in accordance with one embodiment of the disclosure; and

FIG. 2 is an unfold view of a fixation device for pelvis in accordance with one embodiment of the disclosure.

### DETAILED DESCRIPTION OF THE EMBODIMENT

The present invention will be further described with reference to the accompanying drawings.

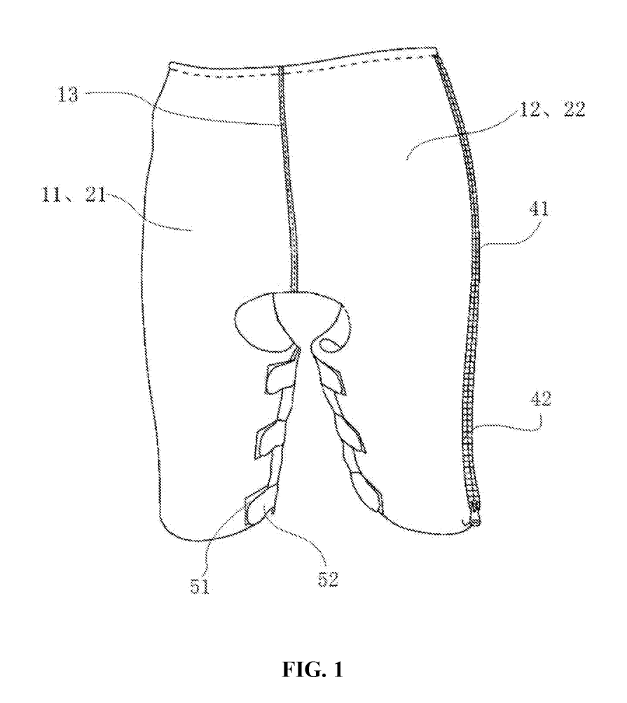

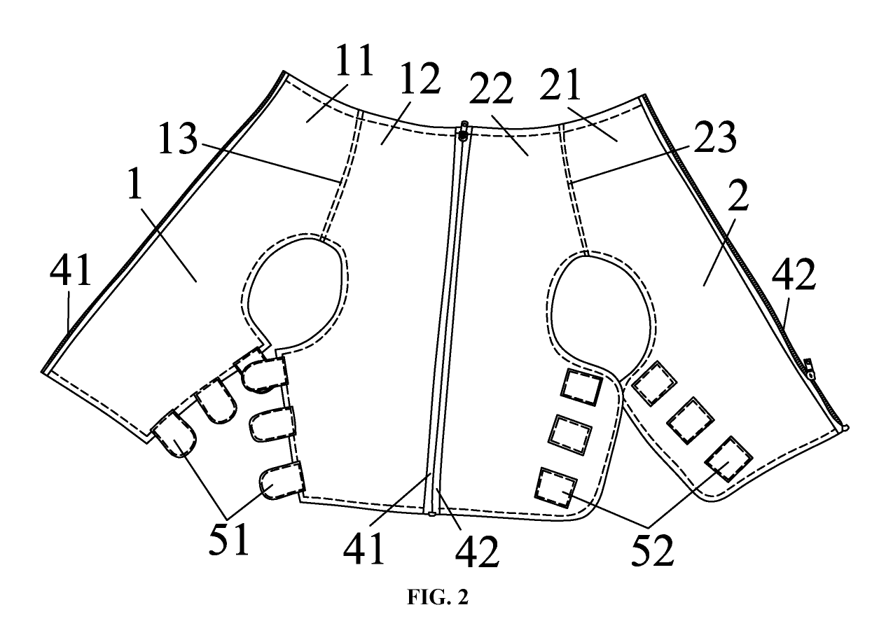

As shown in FIG. 1 and FIG. 2, a fixation device for pelvis comprises a front fixation plate 1 and a rear fixation plate 2. The front fixation plate 1 and the rear fixation plate 2 are detachably connected to each other and form a receiving space between them. When in use, the receiving space is used for receiving the waist, the abdomens, the hip, and the thighs of a patient. The front fixation plate 1 is used to cover the abdomen, and the front side of the waist and thighs. The fixation piece 2 is used to cover the hip and the back side hip of the waist and thighs.

The front fixation plate 1 comprises a first left-side piece 11 and a first right-side piece 12. The first left-side piece 11 comprises a first cavity and a first feeding port. The first right-side piece 12 comprises a second cavity and a second feeding port. The rear fixation plate 2 comprises a second left-side piece 21 and a second right-side piece 22. The second left-side piece 21 comprises a third cavity and a third feeding port. The second right-side piece 22 comprises a fourth cavity and a fourth feeding port. In some embodiments, the cavity is formed by two fabric layers. In particular, the two fabric layers are stacked and overlapped such that the cavity is formed between the overlapped areas of the two fabric layers.

According to the actual situation, the first cavity and the second cavity either communicate with each other or not. The third cavity and the fourth cavity either communicate with each other or not.

In some embodiments, the first cavity and the second cavity do not communicate with each other. The third cavity and the fourth cavity do not communicate with each other. Specifically, the first left-side piece 11 and the first right-side 12 are connected to each other at a position corresponding to the waist and abdomen by sewing, and a first sewing line 13 separates the first cavity and the second cavity so that the first cavity and the second cavity do not communicate with each other. Similarly, the second left-side piece 21 and the second right-side piece 22 are connected to each other at a position corresponding to the waist and the hip by sewing, and a second sewing line 23 separates the third cavity and the fourth cavity so that the third cavity and the fourth cavity do not communicate with each other.

When in use, the polyurethane slurry are injected into the first, second, third and fourth cavities respectively through the first, second, third and fourth feeding ports, and then the polyurethane slurry is processed by the rolling treatment for achieving uniformity. When the polyurethane slurry is at the solid phase softening stage, the fixation device is wrapped around the patient. In this way, when the polyurethane slurry enters the solid stage, the first left-side piece 11, the first right-side piece 12, the second left-side piece 21 and the second right-side piece 22 are solidified and shaped to match the waist, abdomen, the abdomen, and the thighs of the patient. As a result, the shape of the fixation device matches to the patient's abdomen, hip and thighs; and more effective support and fixation is provided to the patient's hip and pelvis. The polyurethane slurry is made of a mixture of a special polyurethane A slurry and a B slurry. The detailed technical solution of the polyurethane slurry is disclosed in U.S. Patent No. 9617370.

In order to allow the polyurethane slurry to be distributed rapidly and evenly throughout the cavity, the cavity is divided into different compartments. In some embodiments, one or more sewing threads are sewed on the first left-side piece 11 according to actual needs to divide the first cavity into a plurality of compartments, and separate feeding ports are disposed on each compartment. The same structure and method is applicable to the first right-side piece 12, the second left-side piece 21 and the second right-side piece 22 to divide the corresponding cavity into a plurality of compartments.

In some embodiments, in order to facilitate the division of the cavity of the first left-side piece 11 into different compartments, different positions of the cavity is provided with feeding ports in advance. Before the polyurethane slurry is injected, the first left-side piece 11 is clamped so as to separate the first cavity into different compartments according to use requirements. Hence, there is no need to determine the number and size of compartments during production. The number and size of different compartments are determined during usage according to actual needs, which provides greater flexibility.

In some embodiments, a hook-and-loop fastener is arranged at the first, the second, the third and the fourth feeding ports. In this way, after the feeding operation is completed, the feeding opening is conveniently closed to prevent the polyurethane slurry from flowing out. The first, second, third and fourth feeding ports are respectively disposed on any positions of the first left-side piece 11, the first right-side piece 12, the second left-side piece 21 and the second right-side piece 22. In some embodiments, the feeding ports are disposed at positions corresponding to the waist.

In order to facilitate the patient to put on or take off the fixation device, the front fixation plate 1 and the rear fixation plate 2 are detachably connected to each other in some embodiments. Specifically, the first left-side piece 11 and the first right-side piece 12 are connected to each other at the positions corresponding to the inner and outer sides of the thighs by the detachable connectors 41, 42, 51, and 52, and the second left-side piece 21 and the second right-side piece 22 are connected to each other at the positions corresponding to the inner and outer sides of the thighs by the detachable connectors 41, 42, 51, 52. In some embodiments, the detachable connectors 41, 42, 51, 52 are zippers, snap fasteners or hook-and-loop fasteners.

In some embodiments, the front fixation plate 1 and the rear fixation plate 2 of the fixation device is made into various sizes for the purpose of fitting various users. The fixation device is easy to use and match to the shape of the waist and hip of the patient, and thus provides effective support to the patient, meets the patient's treatment needs, and shortens the treatment time and rehabilitation cycle.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A fixation device for pelvis, comprising:
(a) a front fixation plate; the front fixation plate comprises a first left-side piece and a first right-side piece; the first left-side piece comprises a first cavity and a first feeding port; the first right-side comprises a second cavity and a second feeding port; and
(b) a rear fixation plate; the rear fixation plate comprises a second left-side piece and a second right-side piece; the second left-side piece comprises a third cavity and a third feeding port; the second right-side panel comprises a fourth cavity and a fourth feeding port; wherein:
the front fixation plate is adapted to cover a front side of a waist and thighs of a patient, and a abdomen of the patient; the rear fixation plate is adapted to cover a back side of the waist and the thighs of the patient, and a hip of the patient;
the front fixation plate and the rear fixation plate are detachably connected to each other to form a space for receiving the waist, the hip and the thighs;
the first left-side piece and the first right-side piece are connected to each other at a position corresponding to the waist and the abdomen;
the second left-side panel and the second right-side panel are connected to each other at a position corresponding to the waist and the hip;
the first, second, third and fourth cavities are respectively disposed in the first left-side piece, the first right-side piece, the second left-side piece and the second right-side piece;
the first, second, third and fourth feeding ports are respectively disposed on the first left-side piece, the first right-side piece, the second left-side piece and the second right-side piece; and
the first, second, third and fourth feeding ports respectively communicate with the first, second, third and fourth cavities; and
when in use, the first, second, third, and fourth cavities are respectively filled with polyurethane slurry through the first, second, third, and fourth feeding ports; and after the polyurethane slurry solidifies, the shapes of the first left-side piece, the first right-side piece, the second left-side piece and the second right-side piece are matched to the waist, the abdomen, the thighs, and the hip.

2. The fixation device of claim 1, wherein:
the fixation device further comprises a detachable connector;
the first left-side piece and the first right-side piece are detachably connected to each other at positions respectively corresponding to an inner side and an outer side of the thighs through the detachable connector;
the second left-side piece and the second right-side piece are detachably connected to each other at positions corresponding to an inner side and an outer side of the thighs through the detachable connector.

3. The fixation device of claim 2, wherein: the detachable connector is a zipper, a snap fastener or a hook-and-loop fastener.

4. The fixation device of claim 1, wherein: the first, the second, the third and the fourth feeding ports are provided with a hook-and-loop fastener.

5. The fixation device of claim 2, wherein:
the first left-side piece, the first right-side piece, the second left-side piece, and the second right-side piece respectively comprises two fabric layers;
the two fabric layers are overlapped; and
the first, second, third and fourth cavities are formed between the two fabric layers.

6. The fixation device of claim 1, wherein:
the first left-side piece is sewed to the first right-side piece so that the first cavity and the second cavity are separate from each other; and
the second left-side piece is sewed to the second right-side piece so that the third cavity and the fourth cavity are separate from each other.

7. The fixation device of claim 1, wherein: the first, the second, the third and the fourth feeding ports are respectively disposed on the first left-side piece, the first right-side piece, the second left-side piece, and the second right-side piece at positions corresponding to the waist.
